(19) United States
(12) Patent Application Publication
(10) Pub. No.: US 2023/0135183 A1
(43) Pub. Date: May 4, 2023

(54) FUNNEL SHAPED SAMPLE RECEIVER

(71) Applicant: Gadi PORATH, Kiriat Tivon (IL)

(72) Inventor: Gadi PORATH, Kiriat Tivon (IL)

(21) Appl. No.: 17/515,579

(22) Filed: Nov. 1, 2021

(51) Int. Cl. B01L 3/00

(52) U.S. Cl. CPC B01L 3/502; B01L 3/5635; B01L 3/567; B01L 2300/048; B01L 2300/042; B01L 2300/0681

(57) ABSTRACT

Disclosed herein, for use with a sample collection vessel for collecting a sample fluid, is a funnel shaped sample receiver having an elongated rigid hollow body with an exterior and an interior, said sample receiver including a funnel shaped wide portion having a wide inlet opening configured for receiving the sample fluid, a narrow portion having a narrow inlet in fluid communication with said wide portion and a narrow outlet to direct the sample fluid into the sample collection vessel, a coupling portion to facilitate latching said wide portion to the sample collection vessel, and wherein said wide inlet opening has a circumference that is greater than a circumference of said narrow inlet, and said wide portion including one or more air release vents to facilitate release of air from the sample collection vessel when the sample fluid drips into the sample collection vessel.

### FIELD OF THE INVENTION

The present disclosure generally relates to sample collection sets.

### BACKGROUND

Field collection of biological samples, such as saliva, allows epidemiologists, medical caretakers, border control agents, or the like to quickly diagnose subjects infected with a serious disease and to prevent the further spread of the disease. For example, during the Covid-19 (SARS-CoV-2) epidemic, it is necessary to identify infected subjects as quickly and efficiently as possible to prevent further spread of the Covid-19 virus by self-collection without requiring potentially infected subjects from travelling to clinics for diagnosis and without risking exposure of the professional staff at the collection site.

### SUMMARY

The following embodiments and aspects thereof are described and illustrated in conjunction with systems, tools and methods which are meant to be exemplary and illustrative, not limiting in scope.

There is provided, in accordance with an embodiment, for use with a sample collection vessel for collecting a sample fluid, a funnel shaped sample receiver having an elongated rigid hollow body with an exterior and an interior, the sample receiver including a funnel shaped wide portion having a wide inlet opening configured for receiving the sample fluid, a narrow portion having a narrow inlet in fluid communication with the wide portion and a narrow outlet to direct the sample fluid into the sample collection vessel, a coupling portion to facilitate latching the wide portion to the sample collection vessel, and the wide inlet opening has a circumference that is greater than a circumference of the narrow inlet, and the wide portion including one or more air release vents to facilitate release of air from the sample collection vessel when the sample fluid drips into the sample collection vessel.

In some embodiments, the narrow outlet comprises a strainer.

In some embodiments, the narrow outlet comprises one or more one separation strips.

In some embodiments, the coupling portion comprises interior threads.

In some embodiments, the narrow portion comprises a width within a range of 3-3.2 millimeters.

There is further provided, in accordance with an embodiment, a sample fluid collection assembly, including a sample collection vessel including a sample vessel inlet disposed at a top portion of the sample collection vessel, a sample collection chamber in fluid communication with the inlet port, a coupling member on an exterior portion of the top portion, a funnel shaped sample receiver having an elongated rigid hollow body with an exterior and an interior, the sample receiver including a funnel shaped wide portion having a wide inlet opening configured for receiving the sample fluid, a narrow portion having a narrow inlet in fluid communication with the wide portion and a narrow outlet to direct the sample fluid into the sample collection vessel, the wide inlet opening has a circumference that is greater than a circumference of the narrow inlet and narrow outlet, and a coupling portion to facilitate latching the funnel shaped sample receiver to the coupling member, the wide portion including one or more air release vents to facilitate release of air from the sample collection chamber when the sample fluid drips into the the sample collection chamber.

There is further provided, in accordance with an embodiment, a sample fluid collection assembly, including sample collection vessel including a sample vessel inlet disposed at a top portion of the sample collection vessel, a sample collection chamber in fluid communication with the inlet port, a coupling member on an exterior portion of the top portion, one or more air vents to facilitate release of air when the sample fluid drips into the sample collection chamber, a funnel shaped sample receiver having an elongated rigid hollow body with an exterior and an interior, the sample receiver including a funnel shaped wide portion having a wide inlet opening configured for receiving the sample fluid, a narrow portion having a narrow inlet in fluid communication with the wide portion and a narrow outlet to direct the sample fluid into the sample collection vessel, the wide inlet opening has a circumference that is greater than a circumference of the narrow inlet and narrow outlet, and the wide portion including a coupling portion to facilitate latching the funnel shaped sample receiver to the coupling member.

In addition to the exemplary aspects and embodiments described above, further aspects and embodiments will become apparent by reference to the figures and by study of the following detailed description.

### BRIEF DESCRIPTION OF THE DRAWINGS

Some non-limiting exemplary embodiments or features of the disclosed subject matter are illustrated in the following drawings.

FIGS. 1A-1B schematically illustrate a sample collection set, according to certain exemplary embodiments;

FIGS. 2A-2B schematically illustrate perspective views of the funnel shaped sample receiver of the sample collection set of FIGS. 1A-1B, according to certain exemplary embodiments;

FIG. 3 schematically illustrates a cross section view of the funnel shaped sample receiver latched to the sample collection vessel of FIGS. 1A-1B, according to certain exemplary embodiments;

FIG. 4 schematically illustrates a close-up cross section view of FIG. 3, according to certain exemplary embodiments;

FIG. 5A schematically illustrate a funnel shaped sample receiver having a filter, according to certain exemplary embodiments; and,

5B schematically illustrate a funnel shaped sample receiver having a strip, according to certain exemplary embodiments.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

References to previously presented elements are implied without necessarily further citing the drawing or description in which they appear.

### DETAILED DESCRIPTION

Disclosed herein is a sample collection assembly, according to certain exemplary embodiments.

In accordance with certain embodiments, the sample collection assembly is configured to facilitate collecting, preserving and testing a biological sample, specifically a sample fluid. As described in more detail below, a sample collection assembly can include a sample collection vessel, a funnel shaped sample receiver, and a vessel cap. In some cases, the sample collection vessel can be pre-loaded with a biological sample preservation reagent, a testing agent, or the like. The funnel shaped sample receiver is attached to the sample collection vessel covering the opening of the sample collection vessel to facilitate dispensing the sample fluid into the sample collection vessel while separating a fluid of the sample fluid from air bubbles mixed therein. The vessel cap facilitates occluding and/or temporarily sealing an opening in the sample collection vessel, thereby preventing the reagent and the sample fluid from spilling out of the sample collection vessel.

Embodiments disclosed herein enable separation of a fluid of the sample fluid from air bubbles in the sample fluid. For example, for detection of the Covid-19 virus in a patient, saliva is collected to for testing to detect the Covid-19 virus. However, saliva includes air bubbles that can interfere with an accurate detection of the Covid-19, therefore, it is required to separate the air bubbles from the fluid of the sample fluid during collection of the sample fluid.

In the context of the present disclosure, without limiting, a sample fluid is/implies saliva or any other biological material that a subject can spit.

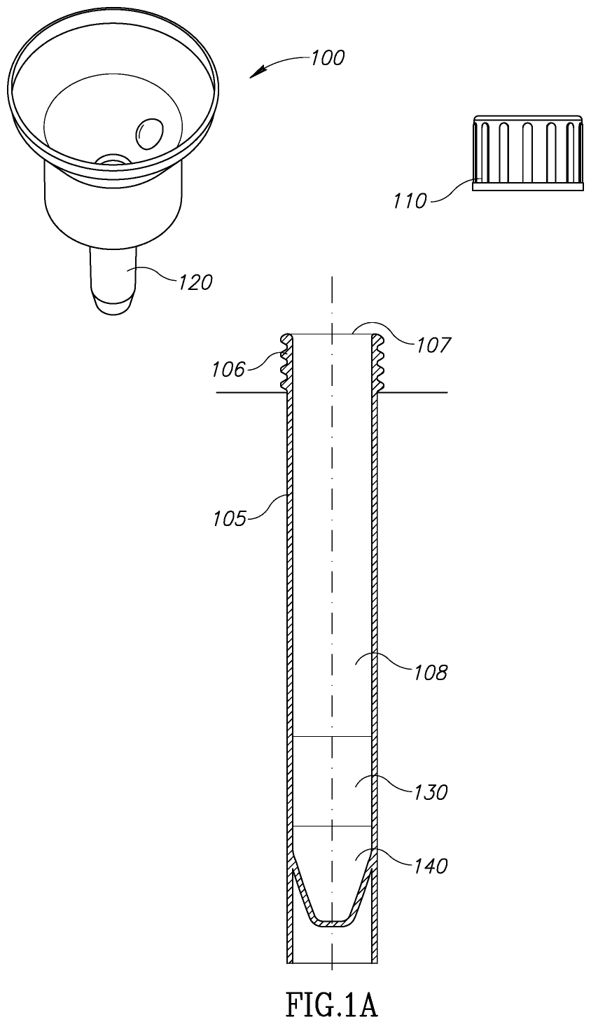

Referring now to FIG. 1A, which schematically illustrates a sample collection assembly or kit 100 including a sample collection vessel 105, a vessel cap 110 and a funnel shaped sample receiver 120, according to certain exemplary embodiments. Sample collection vessel 105 includes a sample vessel opening 107 disposed at a top portion 102 of sample collection vessel 105. Sample collection vessel 105 includes a sample collection chamber 108 configured to store a sample fluid 130. In some embodiments, sample collection chamber 108 can store a preservative reagent or a testing reagent, referenced generally 140, to facilitate preserving the sample fluid 130 or facilitate the immediate testing of sample fluid 130 to determine whether sample fluid 130 contains a predetermined virus, bacteria, or the like. For example, in performance of in-field Polymerase Chain Reaction (“PCR”) testing for Covid-19, the testing reagent 140 facilitates achieving an expedited determination whether a patient is infected with the Covid-19 virus.

Sampled collection vessel 105 includes a connection member 106 disposed on an exterior of top portion 102 to facilitate attaching vessel cap 110 or funnel shaped sample receiver 120 thereto. For example, connection member 106 can include interlocking or mechanically coupling mechanism, such as threads, grooves, or the like.

Referring to FIG. 1B, schematically illustrating funnel shaped sample receiver 120 attached to sample collection vessel 105 to facilitate collection of sample fluid 130, according to certain exemplary embodiments. In some embodiments, sample collection vessel 105 includes a volume indication 109 to inform a sample collector or patient of the volume of sample fluid 130 that is required for accurate testing of sample fluid 130 without bubbles. For example, volume indication 109 can include a minimum or maximum labeling to indicate a minimum or maximum volume of sample fluid 130 that the patient must be disposed into sample collection vessel 105.

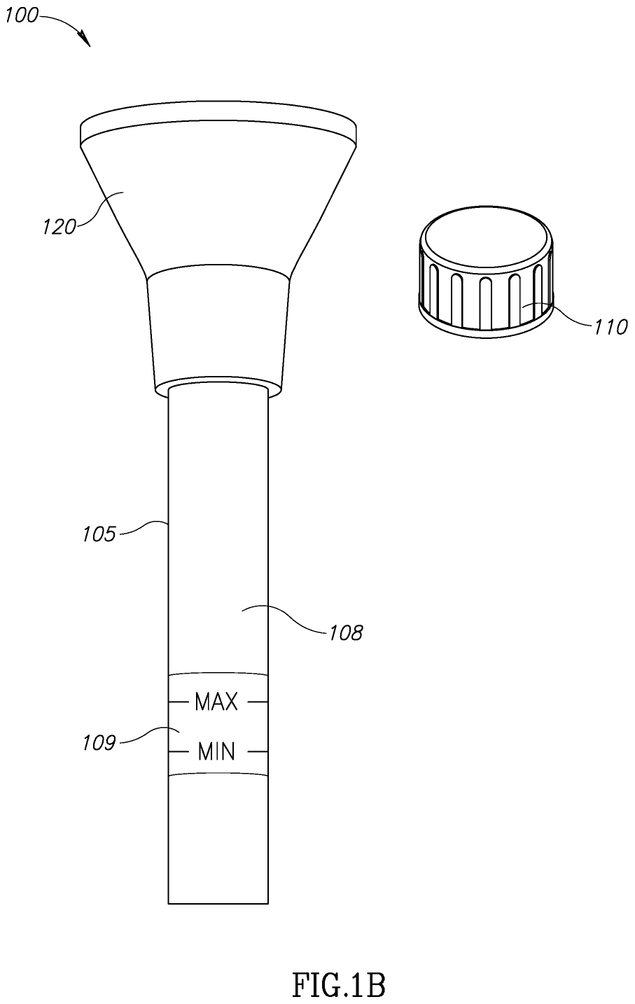

Reference is now made to FIGS. 2A-2B, schematically illustrating funnel shaped sample receiver 120, according to certain exemplary embodiments. Funnel shaped sample receiver 120 includes a funnel shaped wide portion 200 having a wide inlet 202 configured for receiving sample fluid 130 (FIG. 1A). For example, sample fluid 130 is spat into wide inlet 202 by a subject (not shown). In some embodiments, Wide inlet opening 202 has a predetermined circumference to facilitate collecting a maximal volume of sample fluid 130 provided by the subject.

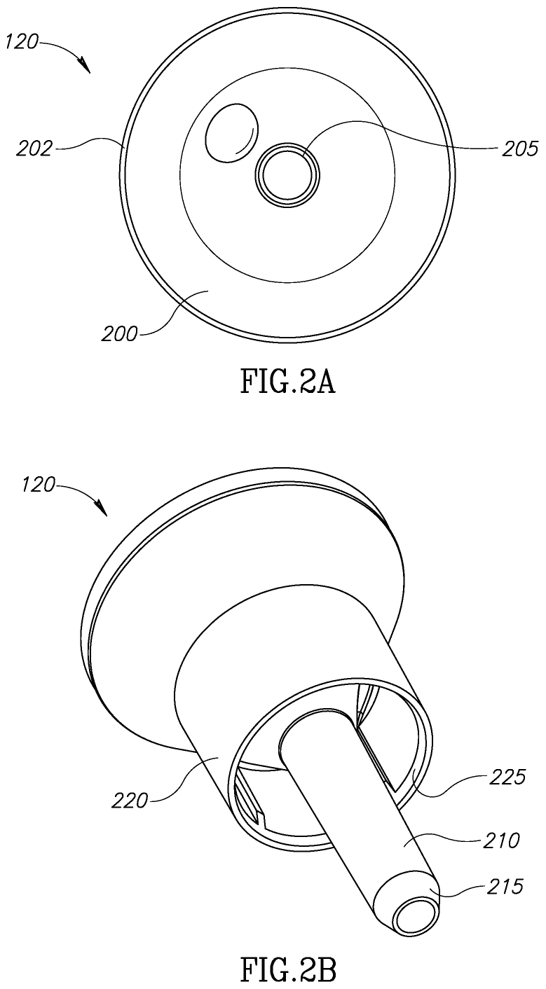

Funnel shaped sample receiver 120 includes a narrow portion 210 having a narrow inlet 205 in fluid communication with funnel shape wide portion 200 and a narrow outlet 215 for directing sample fluid 130 (FIG. 1A) into sample collection vessel 105 (FIG. 1A). Wide inlet opening 202 has a circumference that is greater than a circumference of narrow inlet 205. In some embodiments, narrow inlet 205 and narrow portion 210 can have a circumference within a range of 18.8-20.10 millimeters (“mm”) to control a flow rate of sample fluid 130 into narrow portion 210, thereby preventing air bubbles 315 (FIG. 3) from entering narrow portion 210 with fluid 310 (FIG. 3) of sample fluid 130, as further described in conjunction with FIG. 3. In some embodiments, narrow outlet 215 can have a circumference that is smaller than the circumference of narrow portion 210 to further separate fluid 305 and air bubbles 315.

Funnel shaped sample receiver 120 includes a sealing portion 220 configured to cover sample collection vessel 107 when funnel shaped sample receiver 120 is connected to sample collection vessel 105 (FIG. 1A) thereby preventing the contents of sample collection chamber 108 (FIG. 1A). Sealing portion 220 includes a coupling portion 225 configured to facilitate latching funnel shaped sample receiver 120 to connection member 106 (FIG. 1A). In some embodiments, coupling portion 225 can include interlocking or mechanically coupling mechanisms, such as threads, grooves, or the like.

FIG. 3 schematically illustrates a cross section view of the funnel shaped sample receiver 120 and sample collection vessel 105, according to certain exemplary embodiments. When a new sample fluid 305 is dispensed into funnel shaped wide portion 200, for example, by a subject spitting into Funnel shaped wide portion 200. New sample fluid 305 includes fluid 310 and air bubbles 315 which if allowed to enter sample collection vessel 105 can interfere with an accurate measurement of sample fluid 130 within sample collection vessel 105. In some exemplary embodiments, when sample fluid 130 required for detecting Covid-19, a predetermined volume of saliva is required in the sample fluid 130. Air bubbles 315 in sample fluid 130 can cause interference with accurately testing sample fluid 130 to determine whether the sample fluid 130 contains the Covid-19 virus. Therefore, the sample fluid 130 must be as clear of air bubbles 315 as possible. In some exemplary embodiments, to prevent bubbles, narrow portion 215 can have an opening with a width within a range of 3-3.3 millimeters (“mm”).

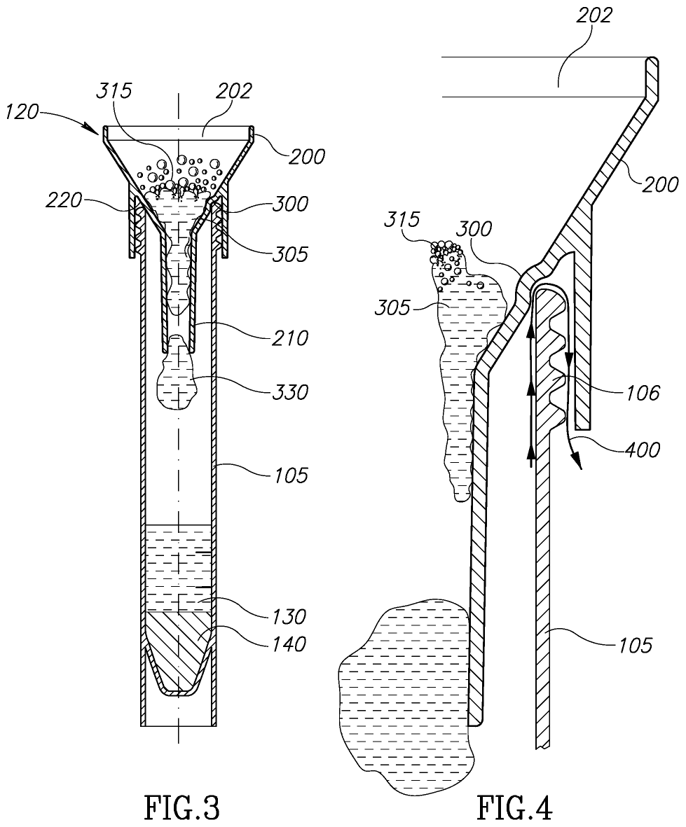

Reference is made to FIG. 4, schematically illustrating a close-up cross section view of funnel shaped sample receiver 120 attached to sample collection vessel 105, according to certain exemplary embodiments. Funnel shaped sample receiver 120 includes one or more air release vents 300 configured to facilitate the release of air 400 when new sample fluid 305 (FIG. 3) drips into the sample collection vessel 105. In some embodiments, air release vent 300 is positioned adjacent to coupling portion 220, for example, between coupling portion 220 and coupling member 106, thereby facilitating the removal of air while preventing spillage of sample fluid 130 (FIG. 1A) and testing reagent 140 (FIG. 1A) from within sample collection vessel 105. In some embodiments, narrow outlet 215 and air release vent 300 along with closed air in sample collection vessel 105 can cause delay in the dripping of the sample fluid 305 and the separation of fluid 310 and air bubbles 315 and can facilitate controlling a drop 330 of sample fluid 305.

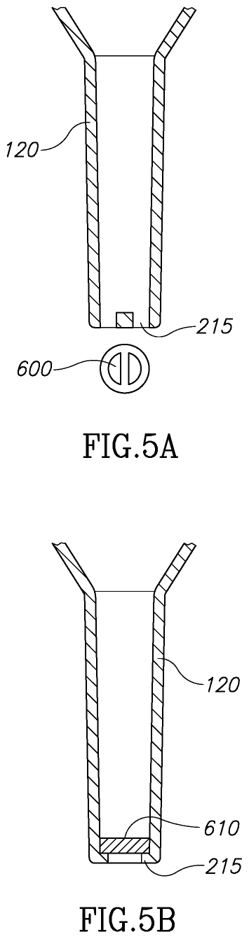

FIG. 5A schematically illustrates narrow outlet 215 having a strip 600, according to certain exemplary embodiments. Strip 600 is configured to further reduce the number of bubbles 315 (FIG. 3) that can pass through narrow outlet 215.

FIG. 5B schematically illustrates narrow outlet 215 having a filter 610, according to certain exemplary embodiments. Filter 610 is configured to further reduce the number of bubbles 315 (FIG. 3) that can pass through narrow outlet 215.

In the context of some embodiments of the present disclosure, by way of example and without limiting, terms such as ‘operating’ or ‘executing’ imply also capabilities, such as ‘operable’ or ‘executable’, respectively. Conjugated terms such as, by way of example, ‘a thing property’ implies a property of the thing, unless otherwise clearly evident from the context thereof.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective. Unless otherwise specified, the terms ‘about’ and/or ‘close’ with respect to a magnitude or a numerical value implies within an inclusive range of −10% to +10% of the respective magnitude or value.

Unless otherwise specified, the terms ‘about’ and/or ‘close’ with respect to a dimension or extent, such as length, implies within an inclusive range of −10% to +10% of the respective dimension or extent. Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values. As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded. Terms in the claims that follow should be interpreted, without limiting, as characterized or described in the specification.

1. For use with a sample collection vessel for collecting a sample fluid, a funnel shaped sample receiver having an elongated rigid hollow body with an exterior and an interior, said sample receiver comprising:
a funnel shaped wide portion having a wide inlet opening configured for receiving the sample fluid;
a narrow portion having a narrow inlet in fluid communication with said wide portion and a narrow outlet to direct the sample fluid into the sample collection vessel;
a coupling portion to facilitate latching said wide portion to the sample collection vessel; and
wherein said wide inlet opening has a circumference that is greater than a circumference of said narrow inlet, and
said wide portion comprising:
at least one air release vent to facilitate release of air from the sample collection vessel when the sample fluid drips into the sample collection vessel.

2. The funnel shaped sample receiver according to claim 1, wherein said narrow outlet comprises a strainer.

3. The funnel shaped sample receiver according to claim 1, wherein said narrow outlet comprises at least one separation strip.

4. The funnel shaped sample receiver according to claim 1, wherein said coupling portion comprises interior threads.

5. The funnel shaped sample receiver of claim 1, wherein said narrow portion comprises a width within a range of 3-3.2 millimeters.

6. A sample fluid collection assembly, comprising:
a sample collection vessel comprising:
a sample vessel inlet disposed at a top portion of said sample collection vessel;
a sample collection chamber in fluid communication with said inlet port;
a coupling member on an exterior portion of said top portion;
a funnel shaped sample receiver having an elongated rigid hollow body with an exterior and an interior, said sample receiver comprising:
a funnel shaped wide portion having a wide inlet opening configured for receiving the sample fluid;
a narrow portion having a narrow inlet in fluid communication with said wide portion and a narrow outlet to direct the sample fluid into the sample collection vessel;
wherein said wide inlet opening has a circumference that is greater than a circumference of said narrow inlet and narrow outlet, and
a coupling portion to facilitate latching said funnel shaped sample receiver to said coupling member;
wherein, said wide portion comprising:
at least one air release vent to facilitate release of air from said sample collection chamber when the sample fluid drips into the said sample collection chamber.

7. A sample fluid collection assembly, comprising:
a sample collection vessel comprising:
a sample vessel inlet disposed at a top portion of said sample collection vessel;
a sample collection chamber in fluid communication with said inlet port;
a coupling member on an exterior portion of said top portion;
at least one air vent to facilitate release of air when the sample fluid drips into said sample collection chamber;
a funnel shaped sample receiver having an elongated rigid hollow body with an exterior and an interior, said sample receiver comprising:
a funnel shaped wide portion having a wide inlet opening configured for receiving the sample fluid;
a narrow portion having a narrow inlet in fluid communication with said wide portion and a narrow outlet to direct the sample fluid into the sample collection vessel;
wherein said wide inlet opening has a circumference that is greater than a circumference of said narrow inlet and narrow outlet, and
said wide portion comprising:
a coupling portion to facilitate latching said funnel shaped sample receiver to said coupling member.
